(19) United States
(12) Patent Application Publication
(10) Pub. No.: US 2026/0110677 A1
(43) Pub. Date: Apr. 23, 2026

(54) SOIL TESTER AND TEST METHOD THEREOF

(71) Applicant: Shenzhen Jinhui Technology Co., Ltd, Shenzhen (CN)

(72) Inventors: Yuanjin HUANG, Shenzhen (CN); Yonghui TANG, Yongzhou (CN)

(73) Assignee: Shenzhen Jinhui Technology Co., Ltd, Shenzhen (CN)

(21) Appl. No.: 19/369,187

(22) Filed: Oct. 25, 2025

(30) Foreign Application Priority Data
Apr. 8, 2025 (CN) 2025206563723

(51) Int. Cl. G01N 33/24; G01N 27/04; G06F 3/147

(52) U.S. Cl. CPC G01N 33/24; G01N 27/041; G06F 3/147

(57) ABSTRACT

The present disclosure provides a soil tester and a test method thereof, and relates to the field of detection instruments. The soil tester includes: a tester main body used for measuring soil characteristics; a sensor circuit used for outputting analog signals reflecting measured results of the soil characteristics; an analog-to-digital converter used for converting the analog signals into digital values; a microcontroller used for receiving and processing the digital values, wherein a nominal value is obtained through initial reading, and the soil tester is provided with an active output circuit to perform the initial reading so as to obtain the nominal value; and a display panel used for displaying measured values of the soil characteristics. In the present disclosure, the analog signals output by the sensor circuit are amplified by an OPA operational amplifier and then directly sent to an ADC pin of the microcontroller.

### CROSS-REFERENCE TO RELATED APPLICATIONS

The application claims priority to Chinese patent application No. 2025206563723, filed on Apr. 8, 2025, the entire contents of which are incorporated herein by reference.

### TECHNICAL FIELD

The present disclosure relates to the field of testing instruments, and more particularly relates to a soil tester and a test method thereof.

### BACKGROUND

Soil environmental monitoring equipment is instrument equipment used for measuring soil environmental parameters, including soil humidity, temperature, pH value, conductivity, and the like. It may assist in soil quality assessment, crop growth monitoring, environmental protection and other work in the fields of agriculture, environmental science, soil science, and the like.

For an existing hand-held soil tester, a detection sensor is inserted into soil by pulling a power cord to perform detection and feedback, but the existing soil tester obtains a nominal value by averaging initial analog readings, and then assigns zero reference related to a neutral value of soil characteristics to the nominal value obtained by averaging the initial readings to obtain a final output result after calibration, that is, an average value serves as a floating standard to be taken as the final output result, and the average value output cannot reflect an actual numerical value.

Therefore, it is necessary to provide a novel soil tester and a test method thereof to solve the above technical problem.

### SUMMARY

In order to solve the above technical problem, the present disclosure provides a soil tester and a test method thereof.

The soil tester provided in the present disclosure includes:
a tester main body used for measuring soil characteristics;
a sensor circuit used for outputting analog signals reflecting measured results of the soil characteristics;
an analog-to-digital converter used for converting the analog signals into digital values;
a microcontroller directly connected to the sensor circuit through an OPA operational amplifier and used for receiving and processing the digital values, wherein the analog signals output by the sensor circuit are amplified by the OPA operational amplifier and then directly sent to an ADC pin of the microcontroller to acquire a plurality of initial readings, and a maximum value of the plurality of initial readings read is taken as a final output result; and
a display panel used for displaying measured values of the soil characteristics.

Further, the tester main body includes a hand-held instrument, a hose and a probing rod, wherein the hand-held instrument is connected to a rear end of the probing rod through the hose;
a front end of the probing rod is a probing end, a first insulation sleeve and a second insulation sleeve are sleeved on a probing end rod body, and the first insulation sleeve and the second insulation sleeve are spaced apart to separate the probing end rod body into the first probing point, the second probing point and the third probing point;
a middle of the probing rod is a through pipe in which a communication wire and the sensor are embedded, the three signal collection ends of the sensor respectively correspond to the first probing point, the second probing point and the third probing point, and each of the signal collection ends is independently connected to the microcontroller; and
the microcontroller is configured to calculate a data maximum value at the three probing points and the data maximum value is displayed through the display panel.

Further, the hose is a metal memory hose with a surface coated with an anti-corrosion layer.

Further, spacing between the first insulation sleeve and the second insulation sleeve is ¼ to ⅓ of a total length of the probing end.

Further, the sensor is an all-in-one composite sensor including a pH detection unit, a humidity detection unit and a conductivity detection unit.

Further, a detection probe of the pH detection unit, a detection probe of the humidity detection unit, and a detection probe of the conductivity detection unit are respectively provided at the first probing point, the second probing point and the third probing point in a corresponding manner.

Further, the display panel is a capacitive touch screen, and the display panel is embedded into a front surface of the hand-held instrument.

Further, a connecting end of the hose with the probing rod is provided with a limiting ring, and a diameter of the limiting ring is greater than that of the hose to prevent the hose from being disconnected from the probing rod.

According to another aspect of the present disclosure, a test method of the soil tester is provided, including the steps of:
step 1, a preparation stage: respectively corresponding three signal collection ends of a sensor to a first probing point, a second probing point and a third probing point;
step 2, data transmission: after the collection ends are inserted into soil, collecting soil parameters at different positions and transmitting the soil parameters to a microcontroller by the three probing points;
step 3, data processing: amplifying analog signals output by a sensor circuit by an OPA operational amplifier and then directly sending the analog signals to an ADC pin of the microcontroller to acquire a plurality of initial readings, taking a maximum value of the plurality of initial readings read as a final output result, and converting the maximum value into a digital value through an analog-to-digital converter; and
step 4, data display: taking a maximum value of three data by the microcontroller and then displaying the maximum value on a display panel, and displaying measured values of soil characteristics.

Compared with the related art, the soil tester and the test method thereof provided in the present disclosure have the following beneficial effects.

1. In the present disclosure, the analog signals output by the sensor circuit are amplified by the OPA operational amplifier and then directly sent to the ADC pin of the microcontroller to acquire a plurality of initial readings, and the maximum value of the plurality of initial readings read is taken as the final output result, so as to change data processing logic and reflect numerical values more authentically.

2. In the present disclosure, during detection, the three probing points synchronously collect parameter values at different soil positions, the microcontroller directly outputs the maximum value from the collected parameter values, without a subtractor or a differential amplification circuit for achieving relative value calculation, and the sensor signals are not dynamically corrected and are displayed through the display panel, so as to solve the problem of large error in conventional single-point detection.

3. In the present disclosure, flexible connection is combined with multi-point data fusion design, which has both operational flexibility and detection reliability, thereby being suitable for rapid soil analysis in farmland, greenhouses and other scenes.

### BRIEF DESCRIPTION OF DRAWINGS

FIG. 1 is a block diagram showing a structure of a soil tester provided in the present disclosure;

FIGS. 2A and 2B is a circuit design diagram 1 of the soil tester provided in the present disclosure;

FIG. 3 is a circuit design diagram 2 of the soil tester provided in the present disclosure;

FIG. 4 is a circuit operation principle diagram of the soil tester provided in the present disclosure;

FIG. 5 is a schematic diagram showing an overall structure of the soil tester provided in the present disclosure;

FIG. 6 is a schematic diagram showing a structure of a probing rod provided in the present disclosure; and

FIG. 7 is a block flow diagram of a test method of the soil tester provided in the present disclosure.

Reference numerals in the drawings: 1. hand-held instrument; 2. hose; 3. probing rod; 4. first insulation sleeve; 5. second insulation sleeve; 6. first probing point; 7. second probing point; 8. third probing point; 9. sensor; 10. display panel; and 11. limiting ring.

### DETAILED DESCRIPTION OF THE EMBODIMENTS

In order to make the objects, technical solutions and advantages of the present disclosure more apparent, an omni-directional rotating soil tester in the present disclosure will now be described in further detail with reference to the accompanying drawings and examples. It should be understood that the specific examples described herein are merely illustrative of the present disclosure and are not intended to limit the present disclosure.

In the description of the present disclosure, unless otherwise specified, the meaning of “a plurality of” is two or more than two; the orientation or positional relationships indicated by the terms “center”, “longitudinal”, “transverse”, “upper”, “lower”, “left”, “right”, “inner”, “outer”, “front end”, “rear end”, “head”, “tail”, “vertical”, “horizontal”, “top”, “bottom”, “internal”, “external”, and the like, are based on the orientation or positional relationships shown in the drawings, are merely for convenience in describing the present disclosure and simplification of the description, do not indicate or imply that the device or element being referred to must have a particular orientation, or be constructed and operated in a particular orientation, and thus should not be construed as limiting the present disclosure. Furthermore, the terms “first”, “second”, “third”, and the like are used for descriptive purposes only and are not to be construed as indicating or implying relative importance.

In describing the present disclosure, it should be noted that unless otherwise expressly specified and limited, the terms “mounted”, “connected with”, and “connected to” should be construed broadly and may, for example, refer to fixed connection, detachable connection, or integral connection; may refer to mechanical connection, or electrical connection; and may refer to direct connection or indirect connection through an intermediate medium, and may refer to internal communication between two elements. The specific meaning of the above terms in this application will be understood by those ordinarily skilled in the art on a case-by-case basis.

### Example 1

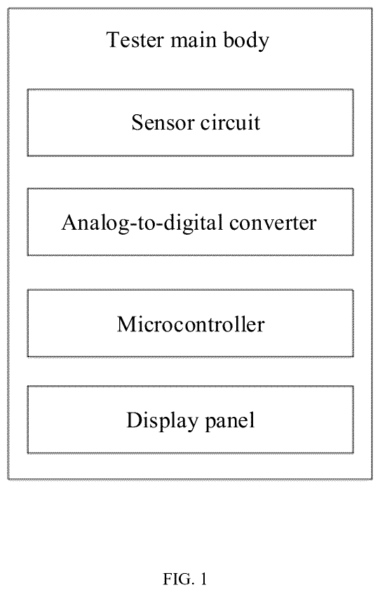

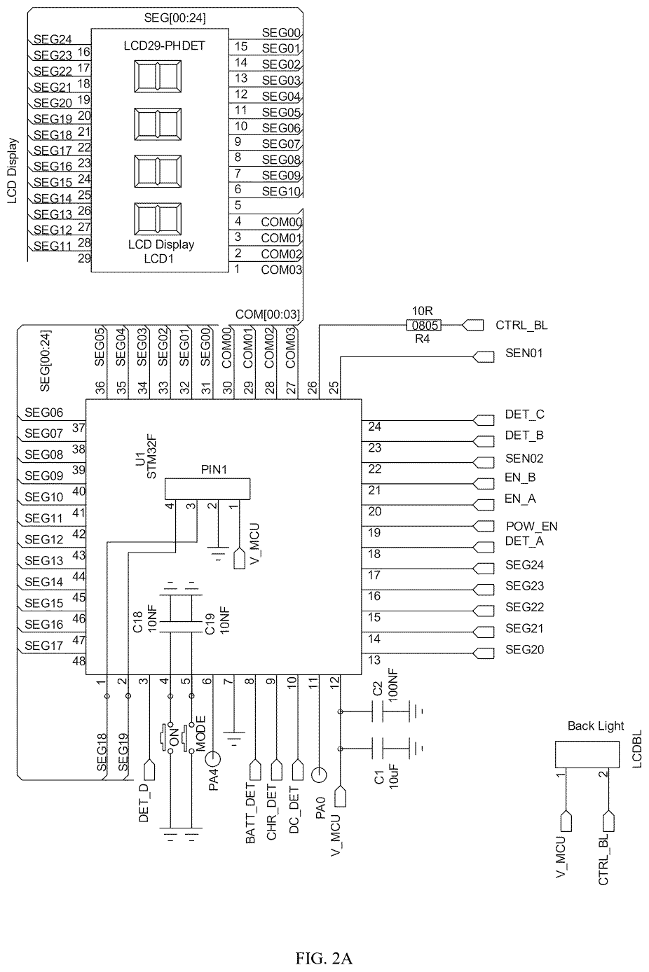

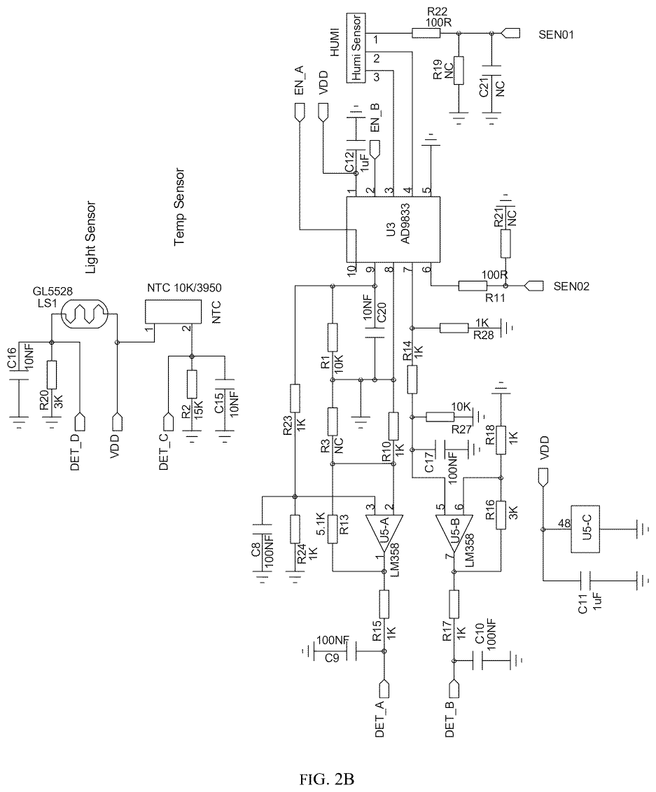

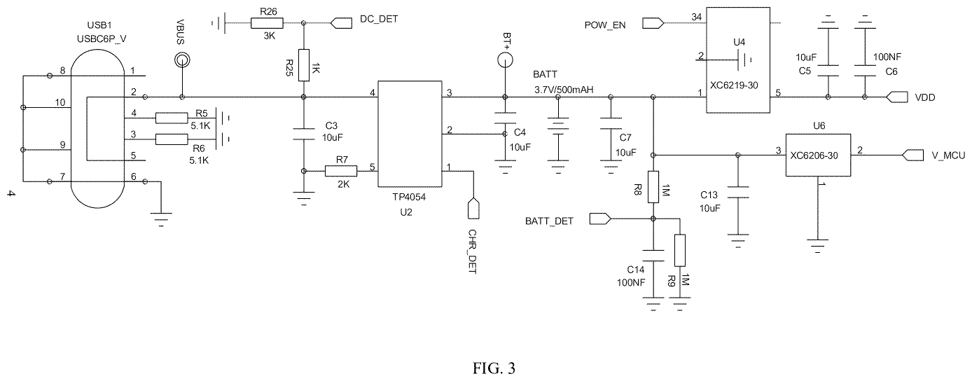

In a specific implementation process, as shown in FIG. 1 to FIG. 6, a soil tester includes:
a tester main body used for measuring soil characteristics;
a sensor circuit used for outputting analog signals reflecting measured results of the soil characteristics;
an analog-to-digital converter used for converting the analog signals into digital values;
a microcontroller directly connected to the sensor circuit through an OPA operational amplifier and used for receiving and processing the digital values, wherein the analog signals output by the sensor circuit are amplified by the OPA operational amplifier and then directly sent to an ADC pin of the microcontroller to acquire a plurality of initial readings, and a maximum value of the plurality of initial readings read is taken as a final output result; and
a display panel used for displaying measured values of the soil characteristics.

The tester main body includes a hand-held instrument 1, a hose 2 and a probing rod 3, wherein the hand-held instrument 1 is connected to a rear end of the probing rod 3 through the hose 2; a front end of the probing rod 3 is a probing end, a first insulation sleeve 4 and a second insulation sleeve 5 are sleeved on a probing end rod body, and the first insulation sleeve 4 and the second insulation sleeve 5 are spaced apart to separate the probing end rod body into the first probing point 6, the second probing point 7 and the third probing point 8; a sensor 9 is provided in the probing rod 3, three signal collection ends of the sensor 9 respectively correspond to the first probing point 6, the second probing point 7 and the third probing point 8, and each of the signal collection ends is independently connected to the microcontroller; and the hand-held instrument 1 includes a display panel, the microcontroller is configured to calculate a data maximum value at the three probing points, and the data maximum value is displayed through the display panel.

The hand-held instrument 1, the hose 2 and the probing rod 3 constitute the tester main body, the hose 2 is made of a bendable and deformable metal memory hose, and a surface of the hose 2 is coated with an anti-corrosion layer, so that a user may bend the hose 2 at will to adjust the orientation of the display panel of the hand-held instrument 1, and data may be directly observed without moving the position of the probing rod; three independent probing points, namely, the first probing point 6, the second probing point 7 and the third probing point 8 are formed at the probing end at the front end of the probing rod 3 through the sleeved first insulation sleeve 4 and second insulation sleeve 5, and a metal rod body at each probing point is electrically connected to three independent signal collection ends of the sensor 9 respectively. After the probing end is inserted into soil, the three probing points collect soil parameters at different positions and transmit the soil parameters to the hand-held instrument 1, and a maximum value of three data is taken by the microcontroller and then the maximum value is displayed on the display panel, so as to reduce random error in single-point detection. A screen may be adjusted at an arbitrary angle: the hose 2 allows 360° rotation of the hand-held instrument 1 relative to the probing rod 3, so as to solve the problem that a conventional rigid rod body cannot flexibly adjust the orientation of the screen; the cooperation of multiple probing points improves the accuracy: measurement error caused by local soil heterogeneity is eliminated by data fusion of three independent probing points, the three metal detection points, namely, the first probing point 6, the second probing point 7 and the third probing point 8 are spaced apart by the first insulation sleeve 4 and the second insulation sleeve 5, the metal conduction is separated by the insulation sleeves and divided into three detection points, and through the signal difference of the three detection points, the middle at the front end of the probing rod 3 is a through pipe in which a communication wire is embedded, and the signals are collected and transmitted to the hand-held instrument, and the microcontroller has a core data processing chip which can accurately calculate a pH value, a temperature value and a humidity value in soil through algorithms.

### Example 2

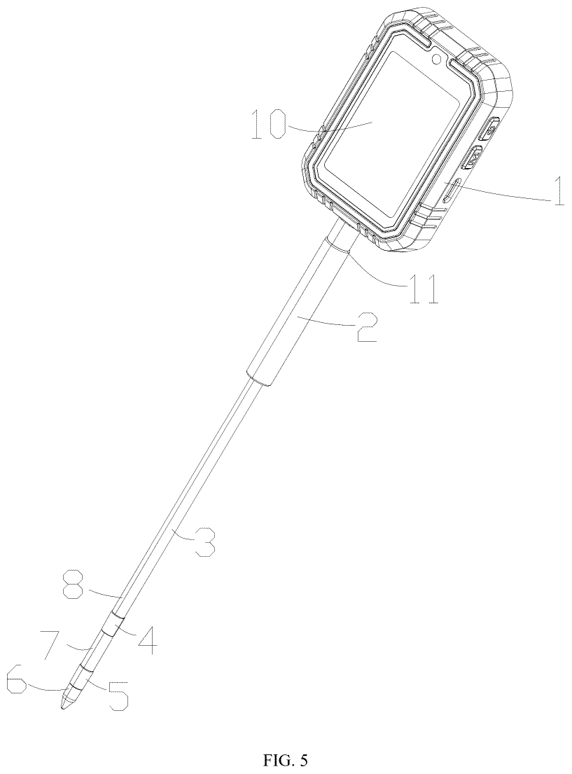

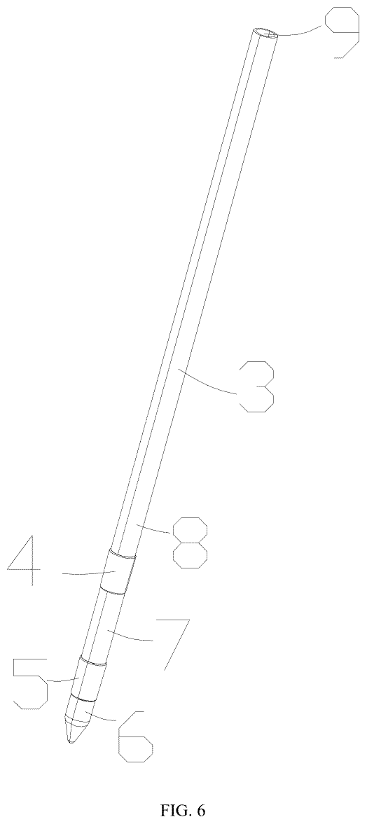

Referring to FIG. 5 and FIG. 6, in another example, the hose 2 is a metal memory hose with a surface coated with an anti-corrosion layer, the hose 2 is made of a nickel-titanium alloy memory metal and may recover an original shape after bending, and the surface of the hose 2 is coated with a silica gel anti-corrosion layer, so as to avoid metal oxidation in saline-alkali soil or a humid environment, and ensure the long-term reliability of flexible connection; by inserting a rear end of the hose 2 into an interface groove of the hand-held instrument 1 and screwing a fixing ring, a front end is threadedly sleeved with a rear end of the probing rod 3, so that the hose 2 is not disconnected or broken in a bending process. Corrosion resistance and durability: the silica gel coating layer is resistant to corrosion by soil chemicals, thereby prolonging the service life of the hose; and adaptive recovery of deformation: the memory metal characteristic ensures that the hose 2 can still remain structural stability after repeated bending.

### Example 3

Referring to FIG. 5 and FIG. 6, in another example, the spacing between the first insulation sleeve 4 and the second insulation sleeve 5 is ¼ to ⅓ of a total length of the probing end. Assuming that the total length of the probing end of the probing rod 3 is L, the spacing between the first insulation sleeve 4 and the second insulation sleeve 5 is set to be in the range of L/4 to L/3 (for example, the spacing is 6-8 cm when the total length is 24 cm), thereby not only ensuring that soil areas covered by each probing point do not overlap, but also avoiding the incapability of being inserted into deep soil due to a too large overall length caused by too large spacing. Reasonable zoning detection: by limiting the spacing between the insulation sleeves, the coverage areas of various probing points are prevented from overlapping or being too far, so as to ensure the diversity and representativeness of data.

### Example 4

Referring to FIG. 5 and FIG. 6, in another example, the sensor 9 is an all-in-one composite sensor including a pH detection unit, a humidity detection unit and a conductivity detection unit.

### Example 5

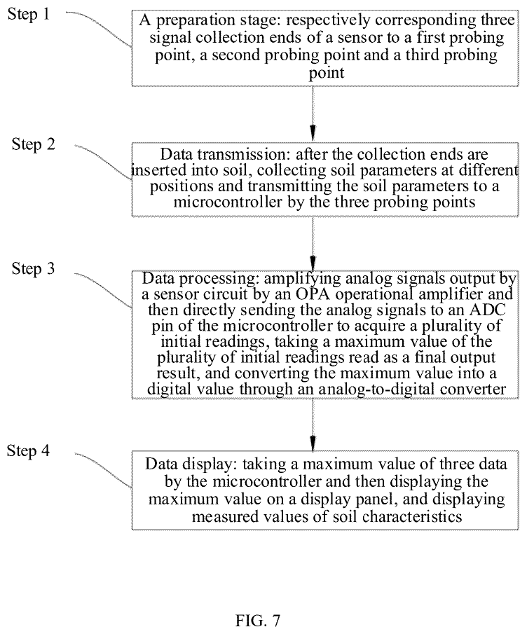

Referring to FIG. 5 and FIG. 7, in another example, a detection probe of the pH detection unit, a detection probe of the humidity detection unit, and a detection probe of the conductivity detection unit are respectively provided at the first probing point 6, the second probing point 7 and the third probing point 8 in a corresponding manner. The sensor 9 is a high-density composite sensor integrated by a pH detection chip, a humidity detection chip and a conductivity detection chip, the probe of the pH detection unit is connected to a metal rod body of the first probing point 6 through a wire, the probe of the humidity detection unit is connected to a metal rod body of the second probing point 7 through a wire, and the probe of the conductivity detection unit is connected to a metal rod body of the third probing point 8 through a wire; and after the probing end is inserted into soil, the three probing points respectively transmit corresponding pH signals, humidity signals and conductivity signals to the hand-held instrument 1 through the metal rod bodies, and the three types of parameters are independently calculated and displayed by the microcontroller respectively. Multi-parameter synchronous detection: the pH value, water content and salt index can be obtained by single insertion operation, thereby improving the detection efficiency; and signal separation anti-interference: different parameter probes are distributed at independent probing points, thereby avoiding data cross-talk caused by electrolysis reaction in a detection process.

### Example 6

Referring to FIG. 5 and FIG. 6, in another example, the display panel is a capacitive touch screen, the touch screen is embedded into a front surface of the hand-held instrument 1, a fully-adhered capacitive touch screen is adopted as the display panel, with a glass panel thereof being bonded to a display screen module through an adhesive layer and then embedded into a groove in the front surface of the hand-held instrument 1, and the periphery thereof being fixed by a sealing rubber ring, and the surface of the touch screen is covered with an anti-scratch coating; and a user switches display modes of detected parameters through touch control operation (for example, only the pH value is displayed or three parameters are simultaneously displayed). Being dustproof and waterproof: the sealing rubber ring prevents soil particles or liquid from penetrating into the screen; and convenient and rapid interaction: conventional keys are replaced with touch operation, thereby reducing the volume and failure rate of the hand-held instrument.

### Example 7

Referring to FIG. 5 and FIG. 6, in another example, a connecting end of the hose 2 with the probing rod 3 is provided with a limiting ring 11, and a diameter of the limiting ring 11 is greater than that of the hose 2 to prevent the hose 2 from being disconnected from the probing rod 3. The limiting ring 11 is of an annular convex structure, with an inner diameter thereof being equal to an outer diameter of the hose 2, and an outer diameter thereof being 2 mm greater than that of the hose 2, and the limiting ring 11 is integrally formed with the hose 2 through an injection molding process; and when the hose 2 is threadedly connected to the probing rod 3, the limiting ring 11 abuts against a rear end interface of the probing rod 3, so as to prevent the hose 2 from falling out of the probing rod 3 due to excessive rotation or pulling; and
mechanical anti-falling design: damage to equipment caused by accidental separation of the hose from the probing rod in an operation process is avoided; and simplified disassembly and assembly: the limiting ring 11 does not affect thread screwing or unscrewing actions while providing an anti-falling function.

### Example 8

Referring to FIG. 7, in another example, a test method of a soil tester, includes the steps of:
step 1, a preparation stage: respectively corresponding three signal collection ends of a sensor 9 to a first probing point 6, a second probing point 7 and a third probing point 8;
step 2, data transmission: after the collection ends are inserted into soil, collecting soil parameters at different positions and transmitting the soil parameters to a microcontroller by the three probing points;
step 3, data processing: amplifying analog signals output by a sensor circuit by an OPA operational amplifier and then directly sending the analog signals to an ADC pin of the microcontroller to acquire a plurality of initial readings, taking a maximum value of the plurality of initial readings read as a final output result, and converting the maximum value into a digital value through an analog-to-digital converter; and
step 4, data display: taking a maximum value of three data by the microcontroller and then displaying the maximum value on a display panel, and displaying measured values of soil characteristics.

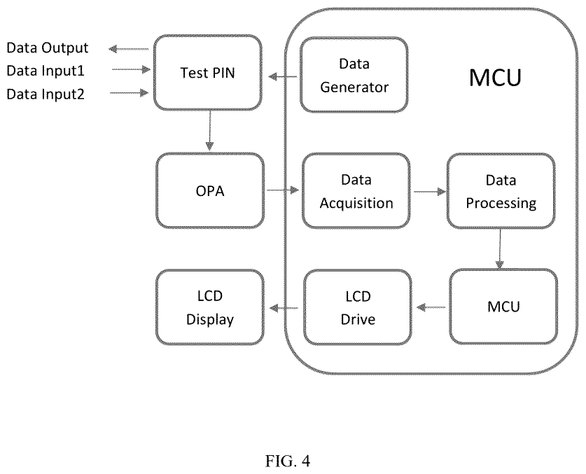

Referring to FIG. 4, during testing, a metal probe head is included, with three electric contacts, one OUT and two Inputs, when equipment runs, a microprogrammed control unit (MCU) instructs to send an electric signal to be output by an OUT PIN needle, and at this moment, the MCU does not receive a feedback signal, and there is only an output signal without an input signal; and if the metal probe is inserted into soil, the electric signal passes through the soil and then is transmitted back through the Input 1 or Input 2, a relevant signal of a corresponding humidity value or pH value is received, the received signal is amplified through the OPA and returned to the MCU to re-identify data, the feedback signal is compared with own output signal by taking the magnitude of a difference value, and a calculation mode is to display a larger value of a comparison result.

The above description is merely preferred examples of the present disclosure and does not constitute limitation on the present disclosure in any form. Although the present disclosure has been disclosed with preferred examples above, it is not intended to limit the present disclosure. Any person skilled in the art may make minor modifications or refinements as equivalent examples with equivalent variations based on the technical content disclosed above, without departing from the scope of the technical solution of the present disclosure. However, all simple modifications, equivalent variations or refinements made to the above examples in accordance with the technical essence of the present disclosure without departing from the content of the technical solution of the present disclosure shall still fall within the scope of the technical solution of the present disclosure.

What is claimed is:

1. A test method of a soil tester, comprising the steps of:
step 1, a preparation stage: respectively corresponding three signal collection ends of a sensor (9) to a first probing point (6), a second probing point (7) and a third probing point (8);
step 2, data transmission: after the collection ends are inserted into soil, collecting soil parameters at different positions by the three probing points, and outputting analog signals and transmitting the analog signals to a microcontroller through a sensor circuit;
step 3, data processing: amplifying the analog signals output by the sensor circuit by an OPA operational amplifier and then directly sending the analog signals to an ADC pin of the microcontroller to acquire a plurality of initial readings, taking a maximum value of the plurality of initial readings read as a final output result, and converting the maximum value into a digital value through an analog-to-digital converter; and
step 4, data display: taking a maximum value of three data by the microcontroller and then displaying the maximum value on a display panel, and displaying measured values of soil characteristics.

2. A soil tester, comprising:
a tester main body used for measuring soil characteristics;
a sensor circuit used for outputting analog signals reflecting measured results of the soil characteristics;
a microcontroller directly connected to the sensor circuit through an OPA operational amplifier and used for receiving and processing digital values, wherein the analog signals output by the sensor circuit are amplified by the OPA operational amplifier and then directly sent to an ADC pin of the microcontroller to acquire a plurality of initial readings, and a maximum value of the plurality of initial readings read is taken as a final output result;
an analog-to-digital converter used for converting the analog signals into the digital values; and
a display panel used for displaying measured values of the soil characteristics.

3. The soil tester according to claim 2, wherein the tester main body comprises a hand-held instrument (1), a hose (2) and a probing rod (3), and the hand-held instrument (1) is connected to a rear end of the probing rod (3) through the hose (2);
a front end of the probing rod (3) is a probing end, a first insulation sleeve (4) and a second insulation sleeve (5) are sleeved on a probing end rod body, and the first insulation sleeve (4) and the second insulation sleeve (5) are spaced apart to separate the probing end rod body into the first probing point (6), the second probing point (7) and the third probing point (8);
a middle of the probing rod (3) is a through pipe in which a communication wire and the sensor (9) are embedded, the three signal collection ends of the sensor (9) respectively correspond to the first probing point (6), the second probing point (7) and the third probing point (8), and each of the signal collection ends is independently connected to the microcontroller; and
the microcontroller is configured to calculate a data maximum value at the three probing points and the data maximum value is displayed through the display panel.

4. The soil tester according to claim 3, wherein the hose (2) is a metal memory hose with a surface coated with an anti-corrosion layer.

5. The soil tester according to claim 3, wherein spacing between the first insulation sleeve (4) and the second insulation sleeve (5) is ¼ to ⅓ of a total length of the probing end.

6. The soil tester according to claim 3, wherein the sensor (9) is an all-in-one composite sensor comprising a pH detection unit, a humidity detection unit and a conductivity detection unit.

7. The soil tester according to claim 6, wherein a detection probe of the pH detection unit, a detection probe of the humidity detection unit, and a detection probe of the conductivity detection unit are respectively provided at the first probing point (6), the second probing point (7) and the third probing point (8) in a corresponding manner.

8. The soil tester according to claim 3, wherein the display panel is a capacitive touch screen, and the display panel is embedded into a front surface of the hand-held instrument (1).

9. The soil tester according to claim 3, wherein a connecting end of the hose (2) with the probing rod (3) is provided with a limiting ring (11), and a diameter of the limiting ring (11) is greater than that of the hose (2) to prevent the hose (2) from being disconnected from the probing rod (3).
